(19) United States
(12) Patent Application Publication
(10) Pub. No.: US 2026/0034514 A1
(43) Pub. Date: Feb. 5, 2026

(54) PRODUCTION OF ORGANIC ACID FROM MONOVALENT ACID SALT VIA ELECTRODIALYSIS USING A THREE-COMPARTMENT ELECTRODIALYSIS UNIT

(71) Applicant: PURAC Biochem B.V., Gorinchem (NL)

(72) Inventors: Cornelis Johannes Govardus VAN STRIEN, Gorinchem (NL); Renaud Benoit MERLET, Gorinchem (NL)

(21) Appl. No.: 19/356,684

(22) Filed: Oct. 13, 2025

Related U.S. Application Data
(63) Continuation of application No. PCT/EP2024/060036, filed on Apr. 12, 2024.

(30) Foreign Application Priority Data
Apr. 14, 2023 (EP) 23167976.2

(51) Int. Cl. B01D 61/44; B01D 61/46; C12P 7/56

(52) U.S. Cl. CPC B01D 61/445; B01D 61/463; B01D 61/465; B01D 61/466; C12P 7/56

(57) ABSTRACT

The present invention provides a process for the production of an organic acid from an aqueous medium of a monovalent salt of an organic acid using bipolar electrodialysis via a three compartment electrodialysis unit.

### CROSS REFERENCE TO RELATED APPLICATIONS

The present application is a continuation of International Application No. PCT/EP2024/060036, filed Apr. 12, 2024, which claims priority to European Patent Application No. 23167976.2, filed Apr. 14, 2023, all of which are hereby incorporated by reference herein in their entireties.

### FIELD OF THE INVENTION

The present invention is in the field of the conversion of organic acid salts to organic acid by means of electrodialysis.

### BACKGROUND OF THE INVENTION

Methods of converting organic acid salts to their organic acids via electrodialysis are described in various publications. The organic acid salt is often obtained via fermentation. During said fermentation usually an alkaline salt is added as neutralizing agent to avoid a pH decrease to such an extent that the micro-organism's activity is inhibited. As a result thereof an organic acid salt is obtained rather than the organic acid itself. Organic acid salt may be converted into its acid via electrodialysis optionally after an intermediate cation exchange reaction and/or a purification step.

WO98/22611 discloses a process for producing organic acids such as lactic acid. The process includes the steps of producing lactic acid by fermentation of a carbohydrate source, resulting in an aqueous fermentation broth containing lactic acid, and adding a calcium base, such as calcium carbonate, to the fermentation broth, thereby producing calcium lactate in the broth. Biomass is removed from the broth, thereby leaving an aqueous solution or dispersion of calcium lactate. The calcium lactate is reacted with a source of ammonium ions, such as ammonium carbonate, or a mixture of ammonia and carbon dioxide, thereby producing an ammonium lactate.

Contaminating cations can be removed by ion exchange. The free lactic acid or a derivative thereof can be separated from the ammonium ions, preferably by salt-splitting electrodialysis.

WO 98/28433 pertains to a method for fermentation of lactic acid from a sugar-containing fermentation liquid in a fermenter by means of lactic acid-fanning bacteria, in which whey protein is present or is added as a nutrient substrate for the lactic acid-fanning bacteria, wherein at least one protease is added to the fermenter during the fermentation, so that hydrolysis of protein to amino acids takes place simultaneously with the fermentation of sugar into organic acid, and wherein lactic acid resulting from the fermentation is isolated from the fermentation liquid. Ammonia is preferably added to result in the formation of ammonium lactate, and lactic acid is preferably isolated by a process comprising ultrafiltration, ion exchange, conventional electrodialysis and electrodialysis with bipolar membranes.

WO2011/95631 discloses a process for the preparation of lactic acid comprising the steps of: a) providing an aqueous medium comprising magnesium lactate; b) adding to the aqueous medium comprising magnesium lactate a monovalent base to form an aqueous medium comprising a water soluble monovalent lactate salt and a solid magnesium base; c) separating the magnesium base from the aqueous medium comprising the water soluble monovalent lactate salt; d) adjusting the concentration of the monovalent lactate salt in the aqueous medium to a value between 10 and 30 wt. %, e) subjecting the aqueous medium comprising the monovalent lactate salt to water-splitting electrodialysis, to produce a first solution comprising monovalent base and a second solution comprising lactic acid and monovalent lactate salt, the electrodialysis being carried out to a partial conversion of 40 to 98 mole %; f) separating the second solution comprising lactic acid and monovalent lactate salt into lactic acid and a solution comprising the monovalent lactate salt by vapor-liquid separation; g) recycling the solution of step f) comprising the monovalent lactate salt to step d).

Despite the various publications describing electrodialysis of organic acid salts, the prior art processes leave much to be desired with respect to either membrane fouling, purity of the product, yield, processing time, water consumption and subsequent water evaporation, or energy consumption over the process as a whole or combinations of these aspects.

Further documents concerning electrolysis are:

US 2008/0272001 which is directed to a method of treating an aqueous stream containing an organic material, the method comprising: supplying a feed stream that includes a target organic desired product, a large molecule/protein material, a soluble mineral material and a non-ionized material to a second compartment of a three-compartment electrodialysis unit; passing the target organic desired product into an adjacent third compartment of the three-compartment electrodialysis unit; passing at least some soluble mineral material into an adjacent first compartment of the three-compartment electrodialysis unit; and passing a remainder of the feed stream through the second chamber.

WO 2020/077917 discloses a multi-stage treatment device for treating high-salt-content waste water is provided, which comprises, a clarifying pool, a softening pool, an ultrafiltration device, a weakly acidic cation bed, a middle-pressure membrane concentration device, a high-pressure membrane concentration device, a nanofiltration device, a first˜ electro-driven membrane device and a bipolar membrane electrodialysis device in sequence.

CN 112237845 discloses a method for preparing an acid system with a pH value of 3.0 to 4.0 by using bipolar membrane electrodialysis technology. Specifically, it involves using brine/seawater as a raw material liquid, and using bipolar membrane electrodialysis technology to produce an acid system with a pH value of 3.0 to 4.0 as a medium for extracting bromine by air blowing.

### SUMMARY OF THE INVENTION

The present invention relates to a process for the production of an organic acid comprising the steps of:
a) providing a feed stream of comprising an aqueous medium of a monovalent salt of an organic acid,
b) splitting the feed stream into at least a first, second and third stream,
c) subjecting the streams to a multi-compartment bipolar electrodialysis comprising an acid flow compartment (1) defined by a bipolar membrane and either a cation or anion selective membrane (6), an intermediate flow department (7) defined by both a cationic- and an anionic selective membrane, and a base flow compartment (2) defined by a bipolar membrane (6) and either a cation or anion selective membrane (6), wherein the compartments are arranged between a positive electrode (anode) (4) and a negative electrode (cathode) (5) and the acid flow compartment (1) and the intermediate flow compartment are separated by a cation selective membrane (6a) and the intermediate flow compartment and the base flow compartment are separated by an anion selective membrane (6b),
whereby the first stream is fed to the acid flow compartment (1), the second stream is fed to the base flow compartment (2), and the third stream is fed to the intermediate flow compartment (7)
d) whereafter an aqueous acid stream comprising an organic acid is leaving the acid flow compartment, an aqueous stream comprising monovalent salt of the organic acid is leaving the intermediate flow compartment and an aqueous base stream comprising a base is leaving the base flow compartment.

Surprisingly it has been found that the present process provides a reduction of the water consumption, while allowing to work in a concentration window for the monovalent salt that is optimal with respect to conductivity.

Optimally step c) comprises subjecting the streams to bipolar electrodialysis in a plurality of acid flow compartments (1), intermediate flow departments (7), and base flow compartments (2), the compartments being present in sets of an acid flow compartment, intermediate flow department (7) and base flow compartment (2) adjacent to each other and said sets being separated from each other by at least a bipolar membrane, forming an electrodialysis unit, whereby the first stream is fed to the acid flow compartments, the second stream is fed to the base flow compartments and the third stream is fed to the intermediate flow compartments.

With this embodiment thus a so-called stack of electrodialysis units is used forming an electrodialysis unit.

Process according to the invention is preferably conducted with the first stream of the aqueous medium of the monovalent salt of the organic acid comprising a volume percentage of at least 25%, preferably at least 33% of monovalent salt of the organic acid based on the total volume of the stream.

Preferably a concentration of the monovalent salt should be maintained so as to ensure that the monovalent salt is in solution. In general the concentration of the monovalent organic salt of the acid in the first and second stream may be chosen to lie between 2% and 99% of the solubility, as measured in grams per liter, of the monovalent organic salt, preferably between 10 and 90% and more preferably between 30-40%.

The process of the present invention was found to be very efficient. The amount of organic acid leaving the acid flow compartment may be between 1-100%, preferably between 85%-99%, more preferably between 92-94% of the molar equivalent of organic salt entering the acid flow compartment.

The aqueous acid stream leaving the acid flow compartment may comprise organic acid in a concentration of at least 20% wt, preferably of at least 25 wt %.

In a further embodiment of the process according to the invention the organic acid is produced in an array of sequentially arranged electrodialysis units whereby the first stream is fed to the acid flow compartment of the first electrodialysis unit and the aqueous acid stream leaving the acid flow compartment of a electrodialysis unit is fed to the acid flow compartment of the subsequent electrodialysis unit, while the second stream is fed to the base flow compartment of the last electrodialysis unit and the aqueous base stream leaving the base flow compartment is fed to the base flow compartment of the previous electrodialysis unit, whereafter an aqueous acid stream comprising an organic acid is leaving the acid flow compartment of the last electrodialysis unit and an aqueous base stream comprising a base and monovalent salt of the organic acid is leaving the base flow compartment of the first electrodialysis unit.

The number of electrodialysis units may vary from 2 to more than 500 units. This set up of more units, also called the stack size, provides the possibility to increase the resulting organic acid concentration in the aqueous acid stream and the metal salt concentration in the aqueous base stream through several stages to obtain the desired concentrations.

Furthermore, the stack size of an electrodialysis unit is limited by the voltage that may be applied across the electrodes. By arranging several electrodialysis units in series as described above, the desired yield and desired end-concentration of both the organic acid and the monovalent salt of the acid can be easily set while minimizing the risk of fouling, lower acid purity and/or current leakage.

Optionally, the aqueous acid stream leaving the last acid flow compartment is further evaporated and distilled under vacuum.

In yet another embodiment of the present invention, at least part of the aqueous base stream leaving the base flow compartment is recycled to the upstream of the feed stream. The recycle stream may for instance be fed to a fermentation step or a cationic exchange reaction step of the organic acid salt production.

The organic acid may be chosen from the group consisting of lactic acid, glycolic acid, malic acid, acetic acid, citric acid, propionic acid, pyruvic acid, oxalic acid, preferably lactic acid.

The monovalent salt may be chosen from the group consisting of sodium, potassium, lithium, ammonium, monoalkyl ammonium, dialkyl ammonium, trialkyl ammonium or tetraalkyl ammonium salt, preferably the monovalent salt is potassium.

The preferred monovalent salt of the organic acid is potassium lactate.

In a further embodiment of the present invention, the aqueous medium comprising a monovalent organic salt of the acid is provided by fermentation, wherein a carbohydrate source is fermented by means of a micro-organism to form an organic acid whereafter a base being added as neutralizing agent during fermentation to provide a bivalent organic salt of the acid which bivalent organic salt of the acid is further converted to a monovalent organic salt of the acid by a double replacement precipitation reaction or an ion exchange step. Suitable carbohydrate sources are known in the art. Examples hereof are sugars such as glucose or sucrose, starch, and the like.

After isolation of the lactic acid further modification or purification steps known in the field may be conducted. Examples are distillation such as vacuum distillation, wiped film evaporation, absorption, extraction, ion exchange, crystallization, conversion to oligo or polymers.

### BRIEF DESCRIPTION OF THE DRAWINGS

FIG. 1 provides a schematic view of a 3-component bipolar electrodialysis unit.

FIG. 2 provides a representation of a possible bipolar electrodialysis process with three stages.

### DETAILED DESCRIPTION

The present invention relates to a process for the production of an organic acid comprising the steps of:
a) providing a feed stream of comprising an aqueous medium of a monovalent salt of an organic acid,
b) splitting the feed stream into at least a first, second and third stream,
c) subjecting the streams to bipolar electrodialysis in an acid flow compartment (1) defined by a bipolar membrane and an anion selective membrane (6b), an intermediate flow department (7) defined by both a cationic selective membrane and an anionic selective membrane, and a base flow compartment (2) defined by a bipolar membrane (6) and a cation selective membrane (6a),
wherein the compartments are arranged between a positive electrode (anode) (4) and a negative electrode (cathode) (5) and the acid flow compartment (1) and the intermediate flow compartment are separated by a cation selective membrane (6a), and the intermediate flow compartment and the base flow compartment are separated by an anion selective membrane (6b),
whereby the first stream is fed to the acid flow compartment (1), the second stream is fed to the base flow compartment (2), and the third stream is fed to the intermediate flow compartment (7)
d) whereafter an aqueous acid stream comprising an organic acid is leaving the acid flow compartment, an aqueous stream comprising monovalent salt of the organic acid is leaving the intermediate flow compartment and an aqueous base stream comprising a base is leaving the base flow compartment.

The process of the present invention provides a reduction of the water consumption, while allowing to work in a concentration window for the monovalent salt of the organic acid that is optimal with respect to conductivity.

The optimal conductivity is determined by the solubility of the monovalent salt of the organic acid, the process conditions used and its restrains, and the specific electrodialysis equipment used. In order to conduct an effective process, the concentration of the feed for the bipolar electrodialysis often has to be increased. In the process according to the present invention, the monovalent salt of the organic acid is also fed to the flow compartment and the intermediate flow compartment whereas in processes according to the prior art the base flow compartment is fed with water, introducing additional water to the system. The water needs to be evaporated at other stages of the process. This is avoided with the process of the present invention and therefore provides an improved water consumption profile.

It is moreover expected that the use of a 3-compartment bipolar electrodialysis unit instead of a 2-compartment bipolar electrodialysis unit may increase the purity of the acid.

The bipolar membrane provides H+ ions in the acid flow compartment and OH− ions in the base flow compartment.

The process may be conducted with both a cationic selective membrane and anionic selective membrane defining the intermediate flow compartment. The cationic ion of the monovalent salt is transferred from the intermediate flow compartment through the cationic selective membrane into the base flow compartment. The organic carboxylate ion moves from the intermediate flow compartment through the membrane into the acid flow compartment.

In an embodiment of the present invention step (c) optimally comprises subjecting the streams to bipolar electrodialysis in a plurality of acid flow compartments (1), intermediate flow departments (7), and base flow compartments (2), the compartments being present in sets of an acid flow compartment, intermediate flow department (7) and base flow compartment adjacent each other and said sets being separated from each other by at least a bipolar membrane, forming an electrodialysis unit, whereby the first stream is fed to the acid flow compartments, the second stream is fed to the base flow compartments and the third stream is fed to the intermediate flow compartments.

With this embodiment thus a so-called stack of electrodialysis units is forming an electrodialysis unit. A bipolar electrodialysis process with multiple units improves the throughput of the process. This stack size is however limited to the maximal voltage that can be applied between two electrodes. A too high voltage will create a risk of current leakage and/or shunting.

The process is usually conducted at a temperature between 20-50° C. Optimally the process is conducted at the highest possible temperature that does not damage the spacers and the membrane of the cells. A spacer as defined in the present invention provides the flow space in the acid flow and base flow channels by separating the membranes from one another, typically in the form of a mesh.

It was found that the process according to the description provides the best yield when it is conducted with the first stream of the aqueous medium of the monovalent salt of the organic acid comprises a volume percentage of at least 25%, preferably at least 33% of monovalent salt of the organic acid of the total volume of the stream.

Preferably a concentration of the monovalent salt should be maintained so as to ensure that the monovalent salt is in solution. In general the concentration of the monovalent organic salt of the acid in the first and second stream may be chosen to lie between 2% and 99% of the solubility, as measured in grams per liter, of the monovalent organic salt preferably between 10 and 90% and more preferably between 30-40%.

The electrodialysis performs best when the feed stream does not contain any insolubles as this may cause fouling of the membranes.

The process was found to be very efficient. The amount of organic acid leaving the acid flow compartment may be between 1-100%, preferably between 85%-99%, more preferably between 92-94% of the molar equivalent of organic salt entering the acid flow compartment. It was found that with a feed stream containing 35 wt % potassium lactate an acid stream could be obtained of 28 wt % of lactic acid.

The aqueous acid stream leaving the acid flow compartment may comprise organic acid in a concentration of at least 20 wt %, preferably of at least 25 wt %.

In a further embodiment of the process according to the present invention the organic acid is produced in an array of sequentially arranged electrodialysis units whereby the first stream is fed to the acid flow compartment of the first electrodialysis unit and the aqueous acid stream leaving the acid flow compartment of a electrodialysis unit is fed to the acid flow compartment of the subsequent electrodialysis unit, while the second stream is fed to the base flow compartment of the last electrodialysis unit and the aqueous base stream leaving the base flow compartment is fed to the base flow compartment of the previous electrodialysis unit, whereafter an aqueous acid stream comprising an organic acid is leaving the acid flow compartment of the last electrodialysis unit and an aqueous base stream comprising a base and monovalent salt of the organic acid is leaving the base flow compartment of the first electrodialysis unit.

The number of electrodialysis units may vary from 2 to more than 500 units. This set up provides the possibility to increase the resulting organic acid concentration in the aqueous acid stream and the metal salt concentration in aqueous base stream the steadily through several stages to obtain the desired concentrations.

As mentioned-above, the amount of units between a pair of electrodes is limited by the voltage that may be applied across the electrodes. With increase of voltage the risk of current leakage and/or shunt increases. This risk is mediated by using an array of sequentially electrodialysis units.

Optionally, the aqueous acid stream leaving the last acid flow compartment is further evaporated and distilled under vacuum.

In another embodiment at least part of the aqueous base stream leaving the base flow compartment is recycled to the feed stream. Recycling part of the base stream avoids having to add water to the feed stream to create the desired acid concentration in the feed stream. This improves the water consumption and subsequent evaporation in the process as a whole. It is expected that the recycle will not detrimentally affect the final purity and yield of the product, while no additional fouling of the membranes is expected.

The organic acid may be chosen from the group consisting of lactic acid, glycolic acid, malic acid, acetic acid, citric acid, propionic acid, pyruvic acid, oxalic acid, preferably the organic acid is lactic acid.

The monovalent salt may be chosen from the group consisting of sodium, potassium, lithium, ammonium, monoalkyl ammonium, dialkyl ammonium, trialkyl ammonium or tetraalkyl ammonium salt, preferably the monovalent salt is potassium. These salts are less prone to foul the membranes and electrodes in the process. Since acids prepared from fermentation are usually in their divalent salt form. The monovalent salts mentioned above can readily be obtained therefrom by a cation-exchange reaction. In one embodiment the aqueous medium comprising a monovalent organic salt of the acid is provided by fermentation, wherein a carbohydrate source is fermented by means of a micro-organism to form an organic acid whereafter a base being added as neutralizing agent during fermentation to provide a bivalent organic salt of the acid which bivalent organic salt of the acid is further converted to a monovalent organic salt of the acid by an ion exchange step.

The description of the drawings below merely serves to illustrate the process according to the description and should not be construed as being limitative to the invention.

### DESCRIPTION OF THE DRAWINGS

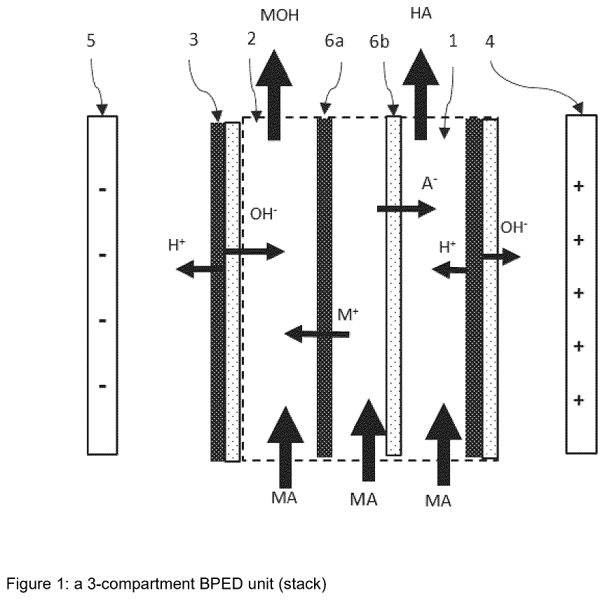

FIG. 1 depicts a 3-component bipolar electrodialysis (BPED) unit (stack), where the dashed line encompasses the repeating cell unit consisting of, from left to right, a base flow compartment, a cation selective membrane, an intermediate flow compartment, an anion selective membrane, an acid flow compartment, and a bipolar membrane. The cell unit(s) are between a cathode, shown as (−) and an anode, shown as (+). Shown entering the intermediate flow compartment is an aqueous solution containing monovalent acid salt (MA); shown leaving the intermediate flow compartment is an aqueous solution containing monovalent acid salt (MA). Shown entering the base flow compartment is an aqueous solution containing monovalent acid salt (MA); shown leaving the base flow compartment is an aqueous solution containing base (MOH). Shown entering the acid flow compartment is a solution containing monovalent acid salt; shown leaving the acid flow compartment is an aqueous solution containing organic acid (HA).

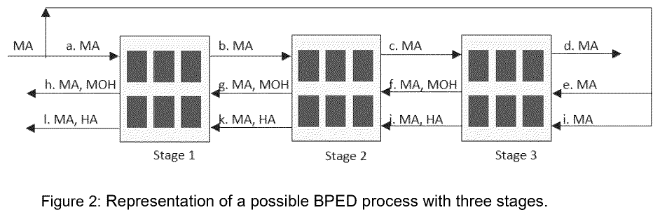

FIG. 2 depicts a possible BPED process with three stages, i.e. having an array of 3 sequentially arranged electrodialysis units. The stages may each have any number of stacks, here represented as rectangles within each stage. At least one stage containing at least one stack is necessary, there are no maximums. The feed stream (1) contains an aqueous solution of a monovalent organic salt (MA) which is split into a first (a) and second (e) stream. Leaving each stage is an aqueous solution of monovalent organic salt (MA) and either organic acid (HA) or base (MOH). Streams a, b, c and d represent streams entering or leaving the acid flow compartments, while streams e, f, g and h represent streams entering or leaving the base flow compartments. The organic acid concentration leaving the acid flow compartments is increased from stage 1 to stage 3. The organic acid salt concentration leaving the acid flow compartments is decreased in concentration from stage 1 to stage 3. The base concentration leaving the base flow compartments is increased from stage 3 to stage 1. The organic salt entering and leaving the base flow compartments is considered inert.

This set up shows that the use of an aqueous solution of a mono salt of the organic acid as a diluent for the feed to the base flow compartment, increases the concentration of the monovalent salt of the organic acid in the final base stream that is recycled upstream of the organic acid production process. As a result, thereof evaporation to the optimal feed concentration for the bipolar electrodialysis may be lowered or avoided altogether.

1. A process for the production of an organic acid, the process comprising:
a) providing a feed stream of comprising an aqueous medium of a monovalent salt of an organic acid,
b) splitting the feed stream into at least a first stream, a second stream, and a third stream,
c) subjecting the streams to bipolar electrodialysis in an acid flow compartment defined by a bipolar membrane and an anion selective membrane, an intermediate flow compartment defined by both a cationic selective membrane and an anionic selective membrane, and a base flow compartment defined by a bipolar membrane and a cation selective membrane,
wherein the compartments are arranged between a positive electrode (anode) and a negative electrode (cathode) and the acid flow compartment, and the intermediate flow compartment are separated by a cation selective membrane and the intermediate flow compartment, and the base flow compartment are separated by an anion selective membrane,
wherein the first stream is fed to the acid flow compartment, the second stream is fed to the base flow compartment, and the third stream is fed to the intermediate flow compartment; and
d) whereafter an aqueous acid stream comprising an organic acid leaves the acid flow compartment, an aqueous stream comprising monovalent salt of the organic acid leaves the intermediate flow compartment, and an aqueous base stream comprising a base leaves the base flow compartment.

2. The process according to claim 1, wherein step c) comprises subjecting the at least first stream, second stream, and third stream to bipolar electrodialysis in a plurality of acid flow compartments, intermediate flow compartments, and base flow compartments; the compartments being present in sets of an acid flow compartment, intermediate flow department, and base flow compartment adjacent to each other; and said sets being separated from each other by at least a bipolar membrane, forming an electrodialysis unit; wherein the first stream is fed to the acid flow compartments, the second stream is fed to the base flow compartments and the third stream is fed to the intermediate flow compartments.

3. The process according to claim 1, wherein the first stream of the aqueous medium of the monovalent salt of the organic acid comprises a volume percentage of at least 25% based on the total feed stream.

4. The process according to claim 1, wherein the concentration of the monovalent organic salt of the organic acid in the first and second stream is between 2% and 99% of the solubility of the monovalent organic salt.

5. The process according to claim 1, wherein the amount of organic acid that leaves the acid flow compartment is between 1-100% of the molar equivalent of organic salt entering the intermediate compartment.

6. The process according to claim 1, wherein the aqueous acid stream that leaves the acid flow compartment comprises organic acid in a concentration of at least 20 wt %.

7. The process according to claim 1, wherein the organic acid is produced in an array of sequentially arranged electrodialysis units whereby the first stream is fed to the acid flow compartment of the first electrodialysis unit and the aqueous acid stream that leaves the acid flow compartment of a electrodialysis unit is fed to the acid flow compartment of the subsequent electrodialysis unit, while the second stream is fed to the base flow compartment of the last electrodialysis unit and the aqueous base stream that leaves the base flow compartment is fed to the base flow compartment of the previous electrodialysis unit, whereafter an aqueous acid stream comprising an organic acid leaves the acid flow compartment of the last electrodialysis unit and an aqueous base stream comprising a base and monovalent salt of the organic acid leaves the base flow compartment of the first electrodialysis unit.

8. The process according to claim 1, further comprising evaporating and distilling under vacuum the aqueous acid stream that leaves the acid flow compartment.

9. The process according to claim 1, further comprising recycling at least part of the aqueous base stream that leaves the base flow compartment upstream of the feed stream.

10. The process according to claim 1, wherein the organic acid is selected from lactic acid, glycolic acid, malic acid, acetic acid, citric acid, propionic acid, pyruvic acid, and oxalic acid.

11. The process according to claim 10, wherein the organic acid is lactic acid.

12. The process according to claim 1, wherein the monovalent salt of the organic acid is selected from sodium, potassium, lithium, ammonium, monoalkyl ammonium, dialkyl ammonium, trialkyl ammonium or tetraalkyl ammonium.

13. The process according to claim 12, wherein the salt is potassium salt.

14. The process according to claim 1, wherein the monovalent salt of the organic acid is potassium lactate.

15. The process according to claim 1, wherein the aqueous medium comprising a monovalent organic salt of the acid is provided by fermentation, wherein a carbohydrate source is fermented by means of a micro-organism to form an organic acid whereafter a base being added as neutralizing agent during fermentation to provide a bivalent organic salt of the acid which bivalent organic salt of the acid is further converted to a monovalent organic salt of the acid by an ion exchange step.
